(19) United States
(12) Patent Application Publication
(10) Pub. No.: US 2025/0095854 A1
(43) Pub. Date: Mar. 20, 2025

(54) METHOD AND SYSTEM FOR CREATING BAYESIAN KNOWLEDGE NETWORKS (BKNS) FOR MEDICAL CONDITIONS

(71) Applicant: Cloudphysician Healthcare Pvt Ltd, Bangalore (IN)

(72) Inventors: Dileep C Unnikrishnan, Palakkad (IN); Dileep Raman, Bengaluru (IN); Dhruv Sud, Bengaluru (IN)

(21) Appl. No.: 18/889,849

(22) Filed: Sep. 19, 2024

(30) Foreign Application Priority Data
Sep. 19, 2023 (IN) 202341062978

(51) Int. Cl. G16H 50/20; G06N 7/01; G16H 10/60; G16H 50/70

(52) U.S. Cl. CPC G16H 50/20; G06N 7/01; G16H 10/60; G16H 50/70

(57) ABSTRACT

This disclosure relates to method and system for creating Bayesian Knowledge Networks (BKNs) for medical conditions. The method includes retrieving an audit trail dataset from an Electronic Medical Record (EMR) corresponding to each of a plurality of patients for one or more medical conditions. The method includes determining a generalized hospital event timeline for each of the one or more medical conditions. The method includes generating a fully connected network of the plurality of nodes of one or more generalized hospital event timelines of the one or more medical conditions. The method includes determining a relation type corresponding to each two of the plurality of nodes of the fully connected network using a causal network learning algorithm. The method includes creating a BKN from the fully connected network based on the determined relation type.

### TECHNICAL FIELD

This disclosure relates generally to medicine, and more particularly to a method and system for creating Bayesian Knowledge Networks (BKNs) for treatment of different medical conditions.

### BACKGROUND

Many healthcare facilities across the world suffer with insufficient human resources and poor infrastructure, putting a significant burden on doctors and medical personnel. The shortage of skilled personnel and essential facilities in hospitals can jeopardize patient care and add additional stress to already overworked medical professionals. In hospitals with limited human resources, doctors often find themselves overwhelmed with a heavy workload. They may have to handle more patients than they can effectively manage, leading to rushed consultations and potential misdiagnoses. At certain times patients also have to wait in long queues due to which the doctors find it difficult to attend to the patients with extensive needs. Moreover, many patients with minor medical issues that can be treated quickly may have to wait for longer durations, leading to worsening of conditions because they didn't receive treatment on time. In a few cases, it has even been observed that patients with critical conditions lose their lives due to unavailability of doctors within the hospital premise.

According to recent studies, 80% of patients with different medical conditions who visit hospital can be treated in a protocolized manner, regardless of whether they visit for general care or intensive care, irrespective of 20% cases that requires supervision of expert doctors. The predefined protocolized manner can automate the treatment process significantly, thereby reducing load of the medical professionals, and dependency of medical experts in case of an emergency. For assisting the medical professionals in providing treatment to the patients, certain probabilistic graphical models can be created and used. One such probabilistic graphical model is Bayesian Knowledge Networks (BKNs) which can reduce workload of the medical professionals by assisting them in managing the treatment process of the patients in a semi-supervised fashion. However, creation of such BKNs manually for various medical conditions may require intensive manual efforts. In other words, the creation of such BKNs manually is resource intensive and untenable.

Therefore, there is the requirement of efficient and reliable technique to create BKNs for various medical conditions.

### SUMMARY

In one embodiment, a method for creating Bayesian Knowledge Networks (BKNs) for medical conditions is disclosed. In one example, the method includes retrieving an audit trail dataset from an Electronic Medical Record (EMR) corresponding to each of a plurality of patients for one or more medical conditions. The audit trail dataset includes a plurality of events. The method further includes determining a generalised hospital event timeline for each of the one or more medical conditions based on the audit trail dataset. The generalised hospital event timeline includes a plurality of nodes corresponding to the plurality of events. The plurality of nodes is connected by unidirected edges in a chronological order of the plurality of events. The method further includes generating a fully connected network of the plurality of nodes of one or more generalised hospital event timelines of the one or more medical conditions. Each two of the plurality of nodes in the fully connected network are interconnected via an undirected edge. The method further includes determining a relation type corresponding to each two of the plurality of nodes of the fully connected network based on the one or more generalised hospital event timelines using a causal network learning algorithm. The method further includes creating a BKN from the fully connected network based on the determined relation type for each two of the plurality of nodes.

In another embodiment, a system for creating Bayesian Knowledge Networks (BKNs) for medical conditions is disclosed. In one example, the system includes a processor and a memory communicatively coupled to the processor. The memory may store processor-executable instructions, which, on execution, may cause the processor to retrieve an audit trail dataset from an Electronic Medical Record (EMR) corresponding to each of a plurality of patients for one or more medical conditions. The audit trail dataset includes a plurality of events. The processor-executable instructions, on execution, further cause the processor to determine a generalised hospital event timeline for each of the one or more medical conditions based on the audit trail dataset. The generalised hospital event timeline includes a plurality of nodes corresponding to the plurality of events. The plurality of nodes is connected by unidirected edges in a chronological order of the plurality of events. The processor-executable instructions, on execution, further cause the processor to generate a fully connected network of the plurality of nodes of one or more generalised hospital event timelines of the one or more medical conditions. Each two of the plurality of nodes in the fully connected network are interconnected via an undirected edge. The processor-executable instructions, on execution, further cause the processor to determine a relation type corresponding to each two of the plurality of nodes of the fully connected network based on the one or more generalised hospital event timelines using a causal network learning algorithm. The processor-executable instructions, on execution, further cause the processor to create a BKN from the fully connected network based on the determined relation type for each two of the plurality of nodes.

In yet another embodiment, a non-transitory computer-readable medium storing computer-executable instructions for creating Bayesian Knowledge Networks (BKNs) for medical conditions is disclosed. In one example, the stored instructions, when executed by a processor, cause the processor to perform operations including retrieving an audit trail dataset from an Electronic Medical Record (EMR) corresponding to each of a plurality of patients for one or more medical conditions. The audit trail dataset includes a plurality of events. The operations further include determining a generalised hospital event timeline for each of the one or more medical conditions based on the audit trail dataset. The generalised hospital event timeline includes a plurality of nodes corresponding to the plurality of events. The plurality of nodes is connected by unidirected edges in a chronological order of the plurality of events. The operations further include generating a fully connected network of the plurality of nodes of one or more generalised hospital event timelines of the one or more medical conditions. Each two of the plurality of nodes in the fully connected network are interconnected via an undirected edge. The operations further include determining a relation type corresponding to each two of the plurality of nodes of the fully connected network based on the one or more generalized hospital event timelines using a causal network learning algorithm. The operations further include creating a BKN from the fully connected network based on the determined relation type for each two of the plurality of nodes.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for creating Bayesian Knowledge Networks (BKNs) for medical conditions, in accordance with some embodiments of the present disclosure.

FIG. 2 illustrates a functional block diagram of various units within a memory of a computing device configured for creating Bayesian Knowledge Networks (BKNs) for medical conditions, in accordance with some embodiments of the present disclosure.

FIG. 3 illustrates a flow diagram of an exemplary process for creating Bayesian knowledge networks (BKNS) for medical record, in accordance with some embodiments of the present disclosure.

FIG. 4 is a table representing an exemplary audit trail dataset retrieved from an EMR of a patient, in accordance with some embodiments of the present disclosure.

FIGS. 5A and 5B represent exemplary generalized hospital event timelines, in accordance with some embodiment of the present disclosure.

FIG. 5C represents an exemplary fully connected network, in accordance with some embodiments of the present disclosure.

FIGS. 6A-6C depict a technique of creation of a BKN from a fully connected network, in accordance with some embodiment of the present disclosure.

FIG. 7 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

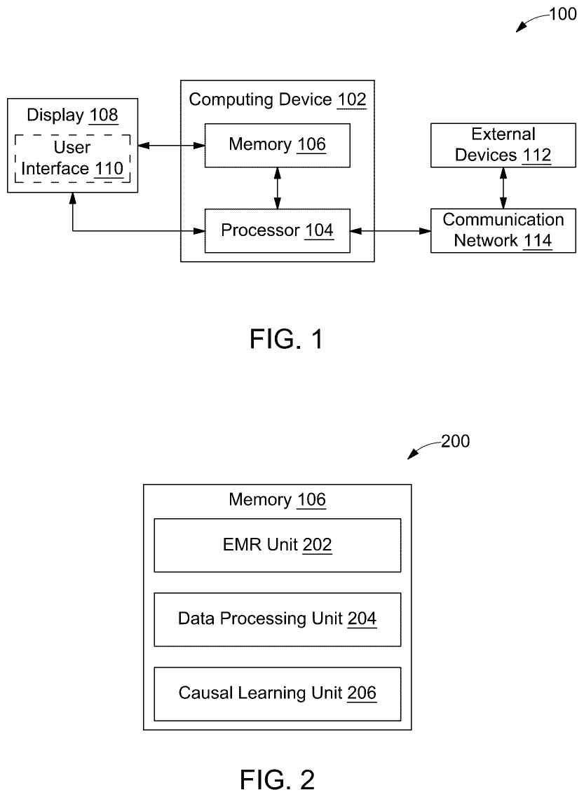

Referring now to FIG. 1, is a block diagram of an exemplary system 100 for creating Bayesian Knowledge Networks (BKNs) for medical conditions is illustrated, in accordance with some embodiments of the present disclosure. The system 100 may include a computing device 102 (for example, server, desktop, laptop, notebook, netbook, tablet, smartphone, mobile phone, or any other computing device), in accordance with some embodiments of the present disclosure. The computing device 102 may create the BKNs for medical conditions. In particular, the computing device 102 may create the BKNs based on medical conditions of different patients.

As will be described in greater detail in conjunction with FIGS. 2-7, the computing device 102 may retrieve an audit trail dataset from an Electronic Medical Record (EMR) corresponding to each of a plurality of patients for one or more medical conditions. The audit trail dataset may include a plurality of events. The computing device 102 may further determine a generalised hospital event timeline for each of the one or more medical conditions based on the audit trail dataset. The generalised hospital event timeline may include a plurality of nodes corresponding to the plurality of events. Further, the plurality of nodes may be connected by unidirected edges in a chronological order of the plurality of events.

The computing device 102 may further generate a fully connected network of the plurality of nodes of one or more generalised hospital event timelines of the one or more medical conditions. In an embodiment, each two of the plurality of nodes in the fully connected network may be interconnected via an undirected edge. The computing device 102 may then determine a relation type corresponding to each two of the plurality of nodes of the fully connected network based on the one or more generalized hospital event timelines using a causal network learning algorithm. Examples of the causal network learning algorithm may include, but is not limited to, Multivariate Information-based Inductive Causation (MIIC) algorithm, Causal Bayesian Network (CBN), causal discovery meta-learning, Causal k-nearest neighbor (CKNN), causal support vector machine (SVM), and causal random forests (RF). Further, the computing device 102 may create a BKN from the fully connected network based on the determined relation type for each two of the plurality of nodes.

In some embodiments, the computing device 102 may include one or more processors 104 and a memory 106. Further, the memory 106 may store instructions that, when executed by the one or more processors 104, cause the one or more processors 104 to create the BKNs for different medical conditions, in accordance with aspects of the present disclosure. The memory 106 may also store various data (for example, the audit trail dataset, the EMR of the plurality of patients, details of different types of medical conditions, and the like) that may be captured, processed, and/or required by the system 100. The memory 106 may be a non-volatile memory (e.g., flash memory, Read Only Memory (ROM), Programmable ROM (PROM), Erasable PROM (EPROM), Electrically EPROM (EEPROM) memory, etc.) or a volatile memory (e.g., Dynamic Random Access Memory (DRAM), Static Random-Access memory (SRAM), etc.).

The system 100 may further include a display 108. The system 100 may interact with a user via a user interface 110 accessible via the display 108. The system 100 may also include one or more external devices 112. In some embodiments, the computing device 102 may interact with the one or more external devices 112 over a communication network 114 for sending or receiving various data. The external devices 112 may include, but may not be limited to, a remote server, a digital device, or another computing system.

Referring now to FIG. 2, a functional block diagram of various units within a memory 106 of a computing device 102 is illustrated, in accordance with some embodiments of the present disclosure. FIG. 2 is explained in conjunction with FIG. 1. The memory 106 of the computing device 102 may include various units, such as, an EMR unit 202, a data processing unit 204, and a causal learning unit 206. Initially, the EMR unit 202 may be configured to receive an EMR corresponding to a plurality of patients as an input. In some embodiments, the EMR corresponding to a patient may be created with time, based on details entered of each action taken for providing a diagnostic treatment based on the medical condition of the patient. The EMR unit 202 may store the EMR of the plurality of patients. The EMR corresponding to the plurality of patients may be for one or more medical conditions. As will be appreciated, for each patient, the EMR of a patient that is stored within the EMR unit 202 may be updated based on a recent action taken during the diagnostic treatment for the medical condition of the patient. Further, the EMR unit 202 may be configured to provide the EMR of the plurality of patients to the data processing unit 204.

The data processing module 204 may be configured to process the EMR of each of the plurality of patients. Further, based on the processing, the data processing module 204 may be configured to retrieve an audit trail dataset from the EMR of each of the plurality of patients. In other words, the data processing module 204 may be configured to retrieve the audit trail dataset from the EMR of each patient with different medical conditions. The audit trail dataset retrieved from the EMR of each patient may include a plurality of events. In addition to the name of each event, the audit trail dataset may include time and date of each event along with the name of a user (e.g., a doctor) who performed that specific event. Examples of the plurality of events may include first visit of a patient to hospital for diagnosis of a disease, first diagnostic session of the patient, details of surgery of the patient, last diagnostic session, discharge of the patient, and the like.

Once the audit trail dataset is retrieved, then the data processing unit 204 may be configured to process the audit trail dataset to determine a generalized hospital event timeline for each of the one or more medical conditions. By way of an example, a generalised hospital event timeline for an orthopedic medical condition (e.g., mild to moderate knee pain) of a patient may include, normal investigation done by a doctor, antibiotics prescribed, result of prescribed antibiotics, and the like. By way of another example, a generalised hospital event timeline for a medical condition, i.e., a lower abdomen pain may include general investigation, test prescribed (such as an X-ray, ultrasound, etc.) based on the investigation, laser stone surgery recommended based on analysis of results of test upon detecting kidney stones, and the like.

The generalised hospital event timeline determined by the data processing module 204 may include a plurality of nodes corresponding to the plurality of events. In other words, each node may correspond to an event of the plurality of events. The plurality of nodes may be connected by unidirected edges in a chronological order of the plurality of events. Further, the data processing module 204 may be configured to generate a fully connected network of the plurality of nodes of one or more generalised hospital event timelines of the one or more medical conditions. In the fully connected network, each two nodes of the plurality of nodes may be interconnected via an undirected edge. Further, the data processing module 204 may send the generated fully connected network to the causal learning unit 206.

The causal learning unit 206 may be configured to determine a relation type corresponding to each two nodes of the plurality of nodes of the fully connected network. The causal learning unit 206 may determine the relation type between each two nodes based on the one or more generalised hospital event timelines using a causal network learning algorithm. In an embodiment, the relation type is one of a causal relation or a non-causal relation. By way of an example, two nodes of the fully connected node are determined to be in the causal relation when a node may directly influence another node. By way of another example, two nodes of the fully connected network are determined to be in the non-causal relation when a node may not directly influence another node.

Upon determining the relation type between each two nodes of the plurality of nodes of the fully connected network, the causal learning unit 206 may be configured to create a BKN from the fully connected network. The BKN may be created based on the determined relation type for each two nodes of the plurality of nodes. In an embodiment, when two nodes are determined to be in causal relation, then the two nodes may be connected via a directed edge in the BKN. In another embodiment, when two nodes are determined to be in non-causal relation, then the two nodes may not be connected via an edge in the BKN.

It should be noted that all such aforementioned units 202-206 may be represented as a single module or a combination of different modules. Further, as will be appreciated by those skilled in the art, each of the units 202-206 may reside, in whole or in parts, on one device or multiple devices in communication with each other. In some embodiments, each of the units 202-206 may be implemented as dedicated hardware circuit comprising custom application-specific integrated circuit (ASIC) or gate arrays, off-the-shelf semiconductors such as logic chips, transistors, or other discrete components. Each of the unit 202-206 may also be implemented in a programmable hardware device such as a field programmable gate array (FPGA), programmable array logic, programmable logic device, and so forth. Alternatively, each of the units 202-206 may be implemented in software for execution by various types of processors (e.g., processor 104). An identified unit of executable code may, for instance, includes one or more physical or logical blocks of computer instructions, which may, for instance, be organised as an object, procedure, function, or other construct. Nevertheless, the executables of an identified unit or component need not be physically located together but may include disparate instructions stored in different locations which, when joined logically together, include the unit and achieve the stated purpose of the unit. Indeed, a unit of executable code could be a single instruction, or many instructions, and may even be distributed over several different code segments, among different applications, and across several memory devices.

As will be appreciated by one skilled in the art, a variety of processes may be employed for creating BKNs for different medical conditions. For example, the exemplary system 100 and the associated computing device 102 may create BKNs for medical conditions by the processes discussed herein. In particular, as will be appreciated by those of ordinary skill in the art, control logic and/or automated routines for performing the techniques and steps described herein may be implemented by the system 100 and the computing device 102 either by hardware, software, or combinations of hardware and software. For example, suitable code may be accessed and executed by the one or more processors on the system 100 to perform some or all of the techniques described herein. Similarly, application specific integrated circuits (ASICs) configured to perform some, or all of the processes described herein may be included in the one or more processors on the system 100.

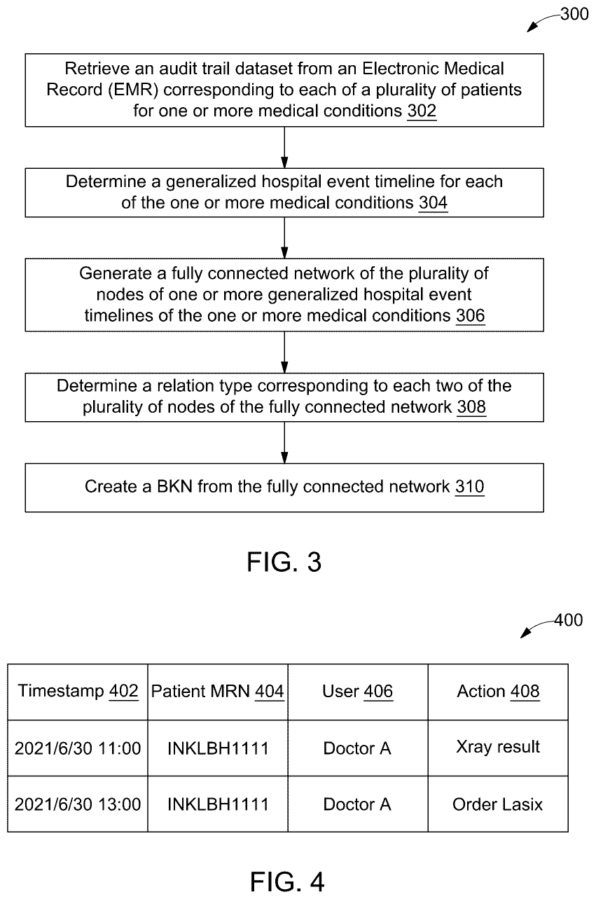

Referring now to FIG. 3, an exemplary process 300 for creating Bayesian Knowledge Networks (BKNs) for medical conditions is illustrated via a flowchart, in accordance with some embodiments of the present disclosure. FIG. 3 is explained in conjunction with FIGS. 1 and 2. The process 300 may be implemented by the computing device 102 of the system 100. At step 302, an audit trail dataset may be retrieved from an Electronic Medical Record (EMR) corresponding to each of a plurality of patients for one or more medical conditions. The audit trail dataset may include a plurality of events. As will be appreciated, the EMR may correspond to a digital version of medical information stored for a patient. The EMR may include the medical treatment history of the patient, such as, disease identified, diagnoses, medications, past surgeries details, immunisation dates, allergies, lab results, doctor's notes, and the like. Further, the EMR of the patient may be used to retrieve the audit trail dataset for the patient. In an embodiment, the audit trail dataset may correspond to a log of actions that happen in the EMR associated with the patient. In addition to the name of each event, the audit trail dataset may include time and date of each event along with the name of a user (e.g., a doctor) who performed that specific event. Examples of the plurality of events may include first visit of a patient to hospital for diagnosis of a disease, first diagnostic session of the patient, details of surgery of the patient, last diagnostic session, discharge of the patient, and the like.

At step 304, a generalised hospital event timeline may be determined. In an embodiment, the generalised hospital event timeline may be determined for each of the one or more medical conditions based on the audit trail dataset. The generalised hospital event timeline may include a plurality of nodes corresponding to the plurality of events. Moreover, the plurality of nodes may be connected by unidirected edges in a chronological order of the plurality of events. In other words, each of the plurality of nodes may be connected by an edge indicating a single direction based on a sequence in which each event of the plurality of events might have occurred. By way of an example, each node of the generalised hospital event timeline may depict an event of the plurality of events, for example, first node may be the first visit of the patient to the hospital, second node may be the medical diagnostics steps taken by the doctor during treatment, third node may be discharge of the patient from the hospital.

Further, at step 306, a fully connected network of the plurality of nodes may be generated. The plurality of nodes may be of one or more generalized hospital event timelines of the one or more medical conditions. In other words, based on each generalised hospital event timeline generated using the corresponding audit trail dataset, the fully connected network may be generated. In an embodiment, each two of the plurality of nodes in the fully connected network may be interconnected via an undirected edge. By way of an example, for different diseases, different treatment procedures may be followed in a hospital based on severity of the patient's medical condition. Based on the followed treatment procedure details of each medical condition, the generalised hospital event timeline may be generated using the audit trail dataset. Further, using the generalised hospital event timeline generated for each medical condition, the fully connected network may be generated.

Once the fully connected network is generated, at step 308, a relation type corresponding to each two of the plurality of nodes of the fully connected network may be determined. In an embodiment, the relation type may be determined based on the one or more generalised hospital event timelines using a causal network learning algorithm. Examples of the causal network learning algorithm may include, but is not limited to, Multivariate Information-based Inductive Causation (MIIC) algorithm, Causal Bayesian Network (CBN), causal discovery meta-learning, Causal k-nearest neighbor (CKNN), causal support vector machine (SVM), and causal random forests (RF). In other words, the causal network learning algorithm may be used for determining relations between each two nodes of the plurality of nodes within the fully connected network. In an embodiment, the relation type between each two nodes may be one of a causal relation or a non-causal relation.

By way of an example, two nodes (e.g., a first node and a second node) of the fully connected node may be determined to be in the causal relation when the first node directly influences the second node. In other words, the first node may be in the causal relation with the second node, when the first node may cause occurrence of the second node. For example, a patient with a smoking habit i.e., determined to be the first node may have higher chances of having lung cancer (i.e., the second node), hence the first node may be in the causal relation with the second node. By way of an example, two nodes of the fully connected node may be determined to be in the non-causal relation when a first node does not directly influence of a second node. For example, a patient with a smoking habit i.e., determined to be the first node may not increase chances of having diabetes (i.e., the second node), hence the first node may be in the non-causal relation with the second node.

Further, at step 310, a BKN may be created from the fully connected network. The BKN may be created based on the determined relation type for each two nodes of the plurality of nodes of the fully connected network. Further, the created BKN may be used for diagnosing patients for plurality of diseases by predicting a treatment from results. In an embodiment, when the relation type of two nodes corresponds to the causal relation, then the two nodes may be connected via a directed edge in the BKN. In another embodiment, when the relation type of two nodes corresponds to non-causal relation, then the two nodes may be not connected via an edge in the BKN.

Once the BKN is created, the created BKN may be rendered on a GUI (same as the UI 110) to the user. Further, the created BKN may be modified based on a user command. In addition, the created BKN may be iteratively updated in real-time. In order to update the BKN in real-time, the audit trail dataset may be retrieved from the EMR in real-time. In other words, any update done in the EMR of the patient may be retrieved and updated in the audit trail dataset in real-time. Further, based on the update in the audit trail dataset, the BKN may be updated in the real-time and presented to the user.

Referring now to FIG. 4, a table 400 representing an exemplary audit trail dataset retrieved from an EMR of a patient is depicted, in accordance with some embodiments of the present disclosure. FIG. 4 is explained in conjunction with FIGS. 1-3. As depicted via the table 400, the audit trail dataset may include the plurality of events. In particular, as depicted via the table 400, along with the plurality of events, the audit trail dataset may include other information, such as a date and a time associated with each event, a patient medical record number (MRN), and name of a doctor.

In the table 400, each row of first column, i.e., ‘timestamp 402’ may represent a date and a time at which an event happened. Each row of the second column, i.e., ‘patient MRN 404’ may represent a medical record number of the patient. Each row of a third column, i.e., ‘user 406’ may represent the name of the doctor who performed the event. Lastly, each row of a fourth column, i.e., ‘action 408’ may represent the name of the event. As will be appreciated, each of the plurality of events within the audit trail dataset may be in the chronological order. By way of an example, as depicted a second row of the table 400, on date 30th June 2021 at 11:00 a.m. (i.e., the timestamp 402) for a patient with MRN, ‘INKLBH1111’ (i.e., the patient MRN 404), a doctor named ‘Doctor A’ (i.e., the user 406) has viewed X-ray results (i.e., the action 408) of the patient. Further, based on the X-ray results of the patient, on 30th June 2021 at 1:00 p.m., the patient was given a Lasix injection by the ‘Doctor A’. Hence, the first event and the second event are in chronological order as the second event, i.e., the Lasix injection given by the ‘Doctor A’, is performed based on analysis of the X-ray report, i.e., the first event.

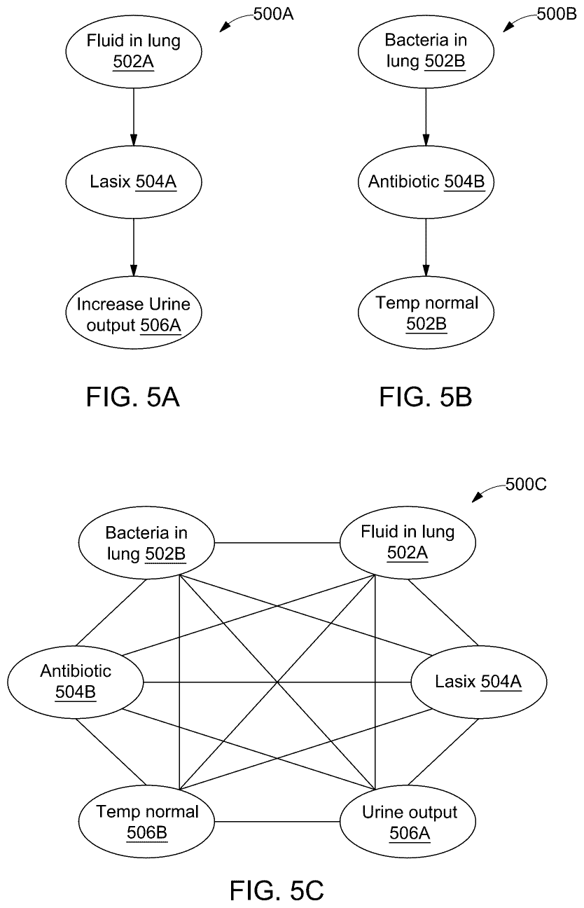

Referring to FIGS. 5A and 5B, represent exemplary generalized hospital event timelines, in accordance with some embodiments of the present disclosure. FIGS. 5A and 5B are explained in conjunction with FIGS. 1-4. Once the audit trail dataset is retrieved from the EMR of the patient as depicted via the table 400 of the FIG. 4, then based on the audit trail dataset, the generalized hospital event timeline may be generated. In FIG. 5A, an exemplary generalized hospital event timeline is depicted via a graph 500A. By way of an example, consider a scenario where the audit trail dataset was retrieved from the EMR of a patient corresponding to treatment given to the patient for edema condition associated with congestive heart failure. Once the audit trail data is retrieved, then the generalized hospital event timeline of the patient treatment for the edema condition may be determined. As explained above in FIGS. 1 to 3, the generalized hospital event timeline may include the plurality of nodes corresponding to the plurality of events.

As depicted via the graph 500A of FIG. 5A, the generalized hospital event timeline determined for the patient with the edema condition may include a set of three nodes, i.e., a node 502A, a node 504A, and a node 506A. Each node of the graphical representation 500A may represent an event. The node 502 may represent an event, ‘fluid in lung’. The event ‘fluid in lung’ may be recorded when based on initial diagnosis (such as analysis of the X-ray results) performed by a doctor (e.g., the doctor A), the fluid may be detected in the lung of the patient. Further, the node 504A may represent an event, ‘Lasix’. The event ‘Lasix’ may be recorded when the Lasix injection was given by the doctor to treat the patient condition, after observing the fluid in the lung of the patient. Further, the node 506A may represent an event, ‘increase urine output’. The event ‘increase urine output’ may be recorded as a result of the Lasix injection given to the patient, based on performed observation.

It should be noted that the Lasix medication is used to remove extra body fluid from the human body, as the result of which the urine output of the patient may have increased. As depicted via the graph 500A, each node of the graph 500A may contain some specific information. Further, each node, i.e., the node 502A, the node 504A, and the node 506A are arranged in a chronological order as per the event and are connected via unidirected edges. In other words, each node of the graph 500A is arranged in a logical sequence for establishing a sequential timeline so that a meaningful interpretation of the treatment process given to the patient may be used for later creating the fully connected network. It should be noted that, for all patients with the edema condition associated with congestive heart failure, the same generalized hospital event timeline may be determined as depicted using the graph 500A.

In FIG. 5B, a graph 500B is another exemplary generalized hospital event timeline determined from the audit trail dataset retrieved from the EMR of a patient with pneumonia condition. The generalized hospital event timeline depicted via the graphical representation 500B may include a set of three events. Each of the set of three events may be represented via a node of the graph 500B. As depicted, the graph 500B may include a set of three nodes for the set of three events. A node 502B may represent an event, i.e., ‘bacteria in lung’, that is recorded based on initial diagnosis (such as analysis of the X-ray results) performed by a doctor. Further, a node 504B may represent an event, ‘antibiotic’, that is recorded when antibiotic may be prescribed to the patient upon detecting bacteria in the lung. A node 506B may represent an event, ‘temperature normal’, that is recorded as a result of the antibiotic given to the patient by the doctor. It should be noted that for all patients with the pneumonia condition, the same generative hospital event timeline may be determined as depicted using the graph 500B.

Referring now to FIG. 5C, an exemplary fully connected network is represented via a graph 500c, in accordance with some embodiments of the present disclosure. FIG. 5C is explained in conjunction with FIGS. 5A and 5B. The graph 500C may be generated using the graph 500A and the graph 500B. The graph 500C depicts a fully connected network generated using the exemplary generalized hospital event timeline determined for the edema condition depicted via the graph 500A and the exemplary generalized hospital event timeline determined for the pneumonia condition depicted via the graph 500B. Further, as depicted via the graph 500C, each two nodes of the fully connected network may be interconnected via an undirected edge.

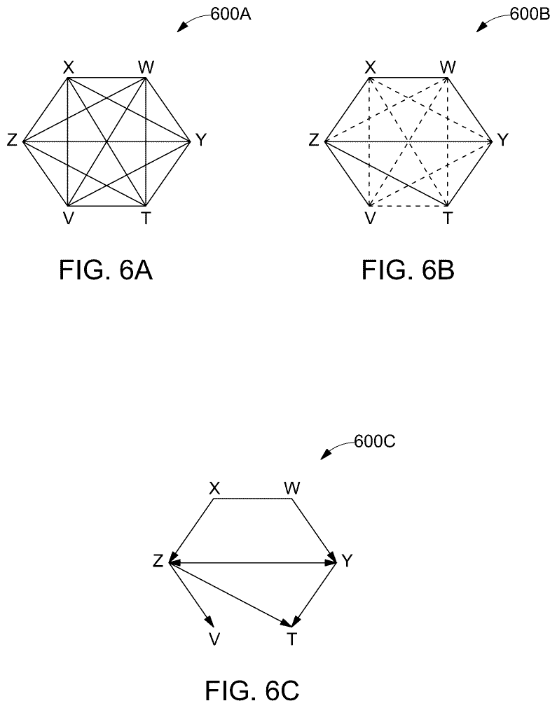

Referring to FIGS. 6A-6C, a technique of creation of a BKN from a fully connected network is depicted, in accordance with some embodiment of the present disclosure. FIGS. 6A-6C is explained in conjunction with FIGS. 5A-5C. With reference to FIG. 1, the technique described in the present FIGS. 6A-6C may be executed by the computing device 102. In FIG. 6A a fully connected network 600A is depicted. With reference to FIG. 50, the fully connected network 600A may correspond to the fully connected network depicted via the graph 500C. As already explained, the fully connected network depicted via the graph 500C may be created for two medical conditions, i.e., the edema condition and the pneumonia condition. The node 502B, the node 504B, and the node 506B may correspond to a node X, a node Z, and a node V respectively. Similarly, the node 502A, the 504A, and the node 506A may correspond to a node W, a node Y, and a node T respectively. As depicted, each two nodes of the fully connected network 600A may be interconnected via the undirected edge.

Further, in order to create the BKN from the fully connected network 600A, dispensable edges may be iteratively removed using the causal network learning algorithm. In order to remove the dispensable edges, the relation type of each two nodes present within the fully connected network 600A may be determined. The relation type may be one of the causal relation or the non-causal relation. Further, based on the determined relation type, each two nodes that are in the non-causal relation may not be connected via the edge as depicted via a network 600B of FIGB. However, each two nodes that are determined to have the causal relation are connected via the undirected edge as depicted in the FIG. 6B. With reference to FIG. 5C, by way of an example, the node 502B, i.e., the node X may not have any association with the node 504A, i.e., the node Y (Lasix injection). In other words, the Lasix injection given for treating the edema (i.e., the fluid in lung) cannot be effective for treating pneumonia. Hence, the edge connecting the node X with node Y may be removed as depicted via a dashed line. It should be noted that, each edge corresponding to each two nodes that are in non-causal relation may be removed as depicted via the dashed lines. With reference to FIG. 2, the relation type between each two nodes may be determined by the causal learning unit 204 using the causal network learning algorithm.

Once each two nodes having the causal relation are determined, then a BKN may be created corresponding to a required medical condition to perform a specific diagnosis. In present FIG. 6, the BKN for two medical conditions, i.e., the edema condition for heart failure, and the pneumonia condition is depicted. In order to create the BKN, each two nodes may be connected via the directed edge based on the causal relation determined between each two nodes, as depicted via a graph 600C of FIG. 6C. In the FIG. 6C, the node X, the node Z, the node V, the node T, and node Y may be in the causal relation. In other words, the occurrence of the event represented via the node X may lead to the occurrence of the event represented via the node Z. Similarly, the occurrence of the event represented by the node Z may lead to the occurrence of the event represented via the node V, the event represented via the node T, and the event represented via the node Y.

In addition, the node W, the node Y, the node T, and the node Z may be in the causal relation. In other words, the occurrence of the event represented via the node W may lead to the occurrence of the event represented via the node Y. Similarly, the occurrence of the event represented by the node Y may lead to the occurrence of the event represented via the node T, and the event represented via the node Z. By way of an example, consider a scenario where a patient with pneumonia condition visits a hospital. In this case, the created BKN may enable the doctor to provide treatment to the patient with the pneumonia condition upon detecting the bacteria in lung (i.e., the node X). For this, the doctor may utilize the created BKN to predict the next step to be done. The next step would be prescribing the antibiotic (i.e., the node Z) for the pneumonia condition. Now, based on the antibiotic prescribed to the patient, there may be three possibilities that may occur. First possibility may be the node V, i.e., the temperature of the person may be become normal. Second possibility may be the node T, i.e., increase in frequency of the urination by the patient, as an effect of the antibiotic. Further, in some cases third possibility, i.e., the node Y may be prescribing Lasix tablet along with the antibiotic to patient to treat high blood pressure and to avoid fluid accumulation in any body part, which in turn may increase the frequency of urination by the patient, i.e., the node T.

As will be appreciated, the directed edges depicted in the graph 600C show all possible directions in which the treatment may proceed upon detecting the pneumonia condition, and the edema condition associated with heart failure. It should be noted that the created BKN may be modified based on the user command (i.e., the doctor). In other words, if the doctor feels that two nodes are in wrong causal relation (i.e., the directed edge is in wrong direction), or should not be in causal relation, then the doctor can either change the direction of connection or delete the connecting edge based on his requirement, by giving the user command corresponding to the two nodes. Similarly, the two nodes can be connected based on the user command given by the doctor.

As will be appreciated, the created BKN may assist the doctor in providing the suitable treatment to the patient for the detected medical condition. It should be noted that, for ease of explanation, the BKN depicted via the graph 600C is used. However, the BKN for these medical conditions, i.e., the pneumonia condition, and the edema condition may be much more complicated. In addition, the BKN may be created for any medical condition using the technique discussed herein.

As will be also appreciated, the above-described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

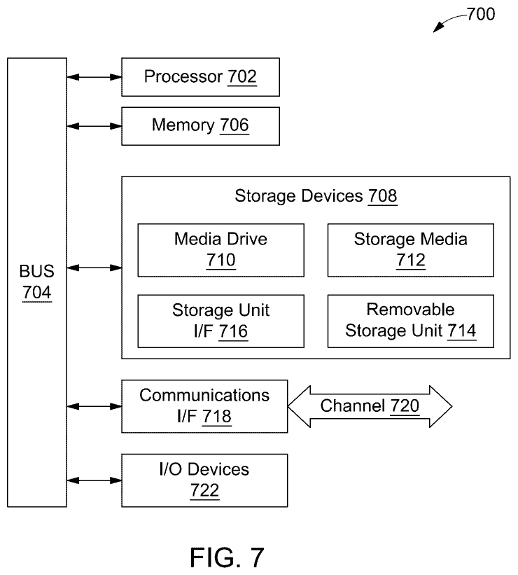

The disclosed methods and systems may be implemented on a conventional or a general-purpose computer system, such as a personal computer (PC) or server computer. Referring now to FIG. 7, an exemplary computing system 700 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 700 may represent, for example, a user device such as a desktop, a laptop, a mobile phone, personal entertainment device, DVR, and so on, or any other type of special or general-purpose computing device as may be desirable or appropriate for a given application or environment. The computing system 700 may include one or more processors, such as a processor 702 that may be implemented using a general or special purpose processing engine such as, for example, a microprocessor, microcontroller, or other control logic. In this example, the processor 702 is connected to a bus 704 or other communication medium. In some embodiments, the processor 702 may be an Artificial Intelligence (AI) processor, which may be implemented as a Tensor Processing Unit (TPU), or a graphical processor unit, or a custom programmable solution Field-Programmable Gate Array (FPGA).

The computing system 700 may also include a memory 706 (main memory), for example, Random Access Memory (RAM) or other dynamic memory, for storing information and instructions to be executed by the processor 702. The memory 706 also may be used for storing temporary variables or other intermediate information during execution of instructions to be executed by the processor 702. The computing system 700 may likewise include a read only memory (“ROM”) or other static storage device coupled to the bus 704 for storing static information and instructions for the processor 702.

The computing system 700 may also include storage devices 708, which may include, for example, a media drive 710 and a removable storage interface. The media drive 710 may include a drive or other mechanism to support fixed or removable storage media, such as a hard disk drive, a floppy disk drive, a magnetic tape drive, an SD card port, a USB port, a micro-USB, an optical disk drive, a CD or DVD drive (R or RW), or other removable or fixed media drive. A storage media 712 may include, for example, a hard disk, magnetic tape, flash drive, or other fixed or removable medium that is read by and written to by the media drive 710. As these examples illustrate, the storage media 712 may include a computer-readable storage medium having stored therein particular computer software or data.

In alternative embodiments, the storage devices 708 may include other similar instrumentalities for allowing computer programs or other instructions or data to be loaded into the computing system 700. Such instrumentalities may include, for example, a removable storage unit 714 and a storage unit interface 716, such as a program cartridge and cartridge interface, a removable memory (for example, a flash memory or other removable memory module) and memory slot, and other removable storage units and interfaces that allow software and data to be transferred from the removable storage unit 714 to the computing system 700.

The computing system 700 may also include a communications interface 718. The communications interface 718 may be used to allow software and data to be transferred between the computing system 700 and external devices. Examples of the communications interface 718 may include a network interface (such as an Ethernet or other NIC card), a communications port (such as for example, a USB port, a micro-USB port), Near field Communication (NFC), etc. Software and data transferred via the communications interface 718 are in the form of signals which may be electronic, electromagnetic, optical, or other signals capable of being received by the communications interface 718. These signals are provided to the communications interface 718 via a channel 720. The channel 720 may carry signals and may be implemented using a wireless medium, wire or cable, fiber optics, or other communications medium. Some examples of the channel 720 may include a phone line, a cellular phone link, an RF link, a Bluetooth link, a network interface, a local or wide area network, and other communications channels.

The computing system 700 may further include Input/Output (I/O) devices 722. Examples may include, but are not limited to a display, keypad, microphone, audio speakers, vibrating motor, LED lights, etc. The I/O devices 722 may receive input from a user and also display an output of the computation performed by the processor 702. In this document, the terms “computer program product” and “computer-readable medium” may be used generally to refer to media such as, for example, the memory 706, the storage devices 708, the removable storage unit 714, or signal(s) on the channel 720. These and other forms of computer-readable media may be involved in providing one or more sequences of one or more instructions to the processor 702 for execution. Such instructions, generally referred to as “computer program code” (which may be grouped in the form of computer programs or other groupings), when executed, enable the computing system 700 to perform features or functions of embodiments of the present invention.

In an embodiment where the elements are implemented using software, the software may be stored in a computer-readable medium and loaded into the computing system 700 using, for example, the removable storage unit 714, the media drive 710 or the communications interface 718. The control logic (in this example, software instructions or computer program code), when executed by the processor 702, causes the processor 702 to perform the functions of the invention as described herein.

Thus, the disclosed method and system try to overcome the technical problem of creating Bayesian Knowledge Networks (BKNs) for medical conditions. The method and system use a causal network learning algorithm for creating the BKN from a fully connected network. The created BKN may assist doctors or any other hospital staff in providing supervision to patients with different medical conditions with minimal human supervision. Further, the created BKN may assist other hospital staff in providing treatment to patients with minor medical conditions and in case of any emergency when the doctor may not be available.

As will be appreciated by those skilled in the art, the techniques described in the various embodiments discussed above are not routine, or conventional, or well understood in the art. The techniques discussed above provide for creating Bayesian Knowledge Networks (BKNs) for medical conditions. The techniques may first retrieve an audit trail dataset from an Electronic Medical Record (EMR) corresponding to each of a plurality of patients for one or more medical conditions. The audit trail dataset may include a plurality of events. The techniques may then determine a generalized hospital event timeline for each of the one or more medical conditions based on the audit trail. The generalized hospital event timeline may include a plurality of nodes corresponding to the plurality of events. The plurality of nodes may be connected by unidirected edges in a chronological order of the plurality of events. The techniques may then generate a fully connected network of the plurality of nodes of one or more generalised hospital event timelines of the one or more medical conditions. Each two of the plurality of nodes in the fully connected network may be interconnected via an undirected edge. The techniques may then determine a relation type corresponding to each two of the plurality of nodes of the fully connected network based on the one or more generalized hospital event timelines using a causal network learning algorithm. The techniques may then create a BKN from the fully connected network based on the determined relation type for each two of the plurality of nodes.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has described method and system for creating Bayesian Knowledge Networks (BKNs) for medical conditions. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for creating Bayesian Knowledge Networks (BKNs) for medical conditions, the method comprising:
retrieving, by a computing device, an audit trail dataset from an Electronic Medical Record (EMR) corresponding to each of a plurality of patients for one or more medical conditions, wherein the audit trail dataset comprises a plurality of events;
determining, by the computing device, a generalized hospital event timeline for each of the one or more medical conditions based on the audit trail dataset, wherein the generalized hospital event timeline comprises a plurality of nodes corresponding to the plurality of events, and wherein the plurality of nodes is connected by unidirected edges in a chronological order of the plurality of events;
generating, by the computing device, a fully connected network of the plurality of nodes of one or more generalized hospital event timelines of the one or more medical conditions, wherein each two of the plurality of nodes in the fully connected network are interconnected via an undirected edge;
determining, by the computing device, a relation type corresponding to each two of the plurality of nodes of the fully connected network based on the one or more generalized hospital event timelines using a causal network learning algorithm; and
creating, by the computing device, a BKN from the fully connected network based on the determined relation type for each two of the plurality of nodes.

2. The method of claim 1, wherein the relation type corresponds to one of a causal relation or a non-causal relation.

3. The method of claim 2, wherein when the relation type of two nodes corresponds to causal relation, the two nodes in the BKN are connected via a directed edge, and wherein when the relation type of two nodes corresponds to non-causal relation, the two nodes in the BKN are not connected by an edge.

4. The method of claim 1, further comprising:
retrieving the audit trail dataset in real-time from the EMR; and
iteratively updating in real-time, the BKN based on the retrieved audit trail.

5. The method of claim 1, further comprising rendering the BKN on a Graphical User Interface (GUI).

6. The method of claim 1, further comprising modifying the BKN based on a user command.

7. A system for creating Bayesian Knowledge Networks (BKNs) for medical conditions, the system comprising:
a processor; and
a memory communicatively coupled to the processor, wherein the memory stores processor-executable instructions, which, on execution, causes the processor to:
retrieve an audit trail dataset from an Electronic Medical Record (EMR) corresponding to each of a plurality of patients for one or more medical conditions, wherein the audit trail dataset comprises a plurality of events;
determine a generalized hospital event timeline for each of the one or more medical conditions based on the audit trail dataset, wherein the generalized hospital event timeline comprises a plurality of nodes corresponding to the plurality of events, and wherein the plurality of nodes is connected by unidirected edges in a chronological order of the plurality of events;
generate a fully connected network of the plurality of nodes of one or more generalized hospital event timelines of the one or more medical conditions, wherein each two of the plurality of nodes in the fully connected network are interconnected via an undirected edge;
determine a relation type corresponding to each two of the plurality of nodes of the fully connected network based on the one or more generalized hospital event timelines using a causal network learning algorithm; and
create a BKN from the fully connected network based on the determined relation type for each two of the plurality of nodes.

8. The system of claim 7, wherein the relation type corresponds to one of a causal relation or a non-causal relation.

9. The system of claim 8, wherein when the relation type of two nodes corresponds to causal relation, the two nodes in the BKN are connected via a directed edge, and wherein when the relation type of two nodes corresponds to non-causal relation, the two nodes in the BKN are not connected by an edge.

10. The system of claim 7, wherein the processor-executable instructions cause the processor to:
retrieve the audit trail dataset in real-time from the EMR; and
iteratively update in real-time, the BKN based on the retrieved audit trail.

11. The system of claim 7, wherein the processor-executable instructions cause the processor to render the BKN on a Graphical User Interface (GUI).

12. The system of claim 7, wherein the processor-executable instructions cause the processor to modify the BKN based on a user command.

13. A non-transitory computer-readable medium storing computer-executable instructions for creating Bayesian Knowledge Networks (BKNs) for medical conditions, the computer-executable instructions configured for:
retrieving an audit trail dataset from an Electronic Medical Record (EMR) corresponding to each of a plurality of patients for one or more medical conditions, wherein the audit trail dataset comprises a plurality of events;
determining a generalized hospital event timeline for each of the one or more medical conditions based on the audit trail dataset, wherein the generalized hospital event timeline comprises a plurality of nodes corresponding to the plurality of events, and wherein the plurality of nodes is connected by unidirected edges in a chronological order of the plurality of events;
generating a fully connected network of the plurality of nodes of one or more generalized hospital event timelines of the one or more medical conditions, wherein each two of the plurality of nodes in the fully connected network are interconnected via an undirected edge;
determining a relation type corresponding to each two of the plurality of nodes of the fully connected network based on the one or more generalized hospital event timelines using a causal network learning algorithm; and
creating a BKN from the fully connected network based on the determined relation type for each two of the plurality of nodes.
